(19) United States
(12) Patent Application Publication
(10) Pub. No.: US 2023/0233153 A1
(43) Pub. Date: Jul. 27, 2023

(54) WEARABLE DEVICE AND METHOD FOR MEASURING BIOMETRIC INFORMATION

(71) Applicant: Samsung Electronics Co., Ltd., Suwon-si (KR)

(72) Inventors: Sungjun KWON, Suwon-si (KR); Jeongmin PARK, Suwon-si (KR); Hyejung SEO, Suwon-si (KR); Choonghee AHN, Suwon-si (KR); Gahee JUNG, Suwon-si (KR); Gunwoo JIN, Suwon-si (KR)

(21) Appl. No.: 18/296,164

(22) Filed: Apr. 5, 2023

Related U.S. Application Data
(63) Continuation of application No. PCT/KR2021/013485, filed on Oct. 1, 2021.

(30) Foreign Application Priority Data
Oct. 23, 2020 (KR) 10-2020-0138444

(51) Int. Cl. A61B 5/00; A61B 5/346; A61B 5/282; A61B 5/256; A61B 5/276; A61B 5/0205

(52) U.S. Cl. CPC A61B 5/7221; A61B 5/346; A61B 5/681; A61B 5/282; A61B 5/7203; A61B 5/256; A61B 5/276; A61B 5/684; A61B 5/6843; A61B 5/0205; A61B 5/02416

(57) ABSTRACT

A wearable device is provided. The wearable device includes a first sensor having a plurality of electrodes, and at least one processor electrically connected to the first sensor. The at least one processor may obtain a first electrocardiogram signal by using a first sensor in a state where the wearable device is worn on a user's body, obtain an electromyogram signal from the first electrocardiogram signal, obtain a second electrocardiogram signal by filtering the electromyogram signal from the first electrocardiogram signal, determine the wearing state of the wearable device on the basis of the intensity of the electromyogram signal and the quality of the second electrocardiogram signal, and output a guide on the wearing state on the basis of a determination result.

### CROSS-REFERENCE TO RELATED APPLICATION(S)

This application is a continuation application, claiming priority under § 365(c), of an International Application No. PCT/KR2021/013485, filed on Oct. 1, 2021, which is based on and claims the benefit of a Korean patent application number 10-2020-0138444, filed on Oct. 23, 2020, in the Korean Intellectual Property Office, the disclosure of which is incorporated by reference herein in its entirety.

### BACKGROUND

### 1. Field

The disclosure relates to a technology for determining a wearing state of a wearable device when biometric information is measured in the wearable device.

### 2. Description of Related Art

An electrocardiography (ECG) measurement method is a method of measuring electrical signals of the autonomic nervous system generated for cardiac muscle movement by using electrodes attached to the skin to observe cardiac activity. A wearable ECG sensor can be used to diagnose some arrhythmias such as atrial fibrillation using simplified electrodes.

Electromyograph (EMG) is an electrical signal generated from skeletal muscle. This is caused by electrical potentials generated when muscle cells are electrically or neutrally activated.

Photoplethysmograph (PPG) is a pulse wave measurement method that can determine the state of heartbeat activity by measuring the amount of blood flowing through blood vessels using the optical characteristics of living tissue. The pulse wave is a pulsating waveform that appears when blood waves in the heart, and can be measured through a change in the blood flow, that is, a change in the volume of the blood vessels, which occurs according to the relaxation and contraction of the heart.

Recently, a technology for measuring a biosignal using a wearable device and examining a person's health condition according to the measurement result is increasing. The wearable device detects an ECG signal at an arbitrary position of the human body and analyzes the detected ECG signal using hardware such as a digital signal processor.

When a user's ECG signal is measured in a wrist-worn wearable device, a stable contact between the electrodes on the rear surface of the wearable device and the skin of the wrist has a great effect on the improvement of the signal quality. For example, when the wearing state of the wearable device is unstable or the skin of the user's wrist is dry, the quality of the ECG signal may deteriorate. Based on the quality of the ECG signal, accuracy of determination of the health state through the ECG signal measurement may be determined.

The above information is presented as background information only to assist with an understanding of the disclosure. No determination has been made, and no assertion is made, as to whether any of the above might be applicable as prior art with regard to the disclosure.

### SUMMARY

When the ECG signal is measured in the wearable device, the deterioration of the signal quality can be significantly improved by attaching the rear surface of the wearable device to the skin of the wrist. Accordingly, in the case of measuring the ECG signal from both hands using the wrist-worn wearable device, when the device is pressed toward the wrist while applying force to the hand holding the device, a rear sensor is in close contact with the skin of the wrist to induce quality improvement.

However, when the device is pressed with more force applied to the hand holding the device than necessary to allow the rear sensor to be in close contact with the skin of the wrist, it may rather cause a problem of deteriorating the signal quality. When force is applied to the hand holding the wearable device, EMG signals are generated from the muscles giving the force. Since the EMG signal is also an electrical signal and the location of the signal generation is close to an ECG sensor, the EMG signal can be sensed together by the ECG sensor. Since the EMG signal is a noise signal from the viewpoint of the ECG signal, a situation in which the signal quality is degraded may occur. In addition, when it is necessary to simultaneously measure the photoplethysmogram from the wrist wearing the device while measuring the ECG signal and, such as a blood pressure estimation technology based on pulse wave transit time, an excessively closely attached sensor may interfere with the observation of changes in the volume of blood vessels, thereby degrading the quality of a photoplethysmogram signal.

Aspects of the disclosure are to address at least the above-mentioned problems and/or disadvantages and to provide at least the advantages described below. Accordingly, an aspect of the disclosure is to provide a technology for determining a wearing state of a wearable device when biometric information is measured in the wearable device.

Additional aspects will be set forth in part in the description which follows and, in part, will be apparent from the description, or may be learned by practice of the presented embodiments.

In accordance with an aspect of the disclosure, a wearable device is provided. The wearable device includes a first sensor having a plurality of electrodes, and at least one processor configured to be electrically connected to the first sensor, wherein the at least one processor may be configured to obtain a first electrocardiogram (ECG) signal using the first sensor while the wearable device is worn on a user's body, obtain an electromyograph (EMG) signal from the first ECG signal, obtain a second ECG signal by filtering the EMG signal from the first ECG signal, determine a wearing state of the wearable device based on an intensity of the EMG signal and a quality of the second ECG signal, and output a guide on the wearing state based on a result of the determination.

In accordance with another aspect of the disclosure, a method of operating a wearable device is provided. The method includes obtaining a first ECG signal using a first sensor while the wearable device is worn on a user's body, obtaining an EMG signal from the first ECG signal, obtaining a second ECG signal by filtering the EMG signal from the first ECG signal, and determining a wearing state of the wearable device based on an intensity of the EMG signal and a quality of the second ECG signal.

In the case of measuring biometric information, a wearable device according to various embodiments according to the disclosure may improve the accuracy of measuring the biometric information by guiding a wearing state when a user attaches the wearable device to the user's body with excessive force or affects ECG measurement with unnecessary muscle activity.

A wearable device according to various embodiments may improve signal quality without a change in the size of the wearable device by analyzing the wearing state through the intensity of an EMG signal mixed with an ECG signal without a separate sensor.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram illustrating mounting of a wearable device on a part of a body according to an embodiment of the disclosure;

FIG. 2 is a perspective view illustrating a wearable device according to an embodiment of the disclosure;

FIG. 3 is a block diagram illustrating a wearable device according to an embodiment of the disclosure;

FIG. 4 is a flowchart illustrating determining of a wearing state of a wearable device in the wearable device according to an embodiment of the disclosure;

FIG. 5 is a flowchart illustrating determining of a wearing state of a wearable device in the wearable device according to an embodiment of the disclosure;

FIG. 6 is a flowchart illustrating outputting of guides on a plurality of wearing states in a wearable device according to an embodiment of the disclosure;

FIG. 7 is a diagram illustrating an electrocardiogram (ECG) signal obtained through a wearable device according to an embodiment of the disclosure;

FIG. 8 is a diagram illustrating a photoplethysmography signal obtained through a wearable device according to an embodiment of the disclosure;

FIG. 9 is a diagram illustrating a signal obtained by separating an EMG signal from an ECG signal obtained through a wearable device according to an embodiment of the disclosure;

FIG. 10 is a diagram illustrating an ECG measurement user interface (UI) displayed on a display in a wearable device according to an embodiment of the disclosure;

FIG. 11 is a diagram illustrating a wearing state guide UI displayed on a display in a wearable device according to an embodiment of the disclosure;

FIG. 12 is a diagram illustrating a plurality of guide UIs for a wearing state displayed on a display in a wearable device according to an embodiment of the disclosure; and

FIG. 13 is a block diagram illustrating an electronic device in a network environment according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

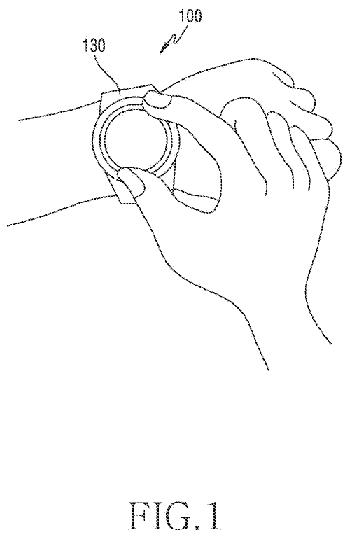

FIG. 1 is a diagram illustrating mounting of a wearable device on a part of a body according to an embodiment of the disclosure.

According to an embodiment, a wearable device 100 of FIG. 1 may be a smart watch as shown. Without being limited thereto, the wearable device 100 may be various types of devices that can be used by being attached to a part of the user's body.

Referring to FIG. 1, according to an embodiment, the wearable device 100 may include a strap 130, and the strap 130 may be attached to the user's body by being wound around the user's wrist. Without being limited thereto, the wearable device 100 may be attached to various parts of the user's body according to the shape, size, and/or other various factors of the wearable device 100. For example, the wearable device 100 may be attached to the hand, back of the hand, fingers, fingernails, and/or fingertips.

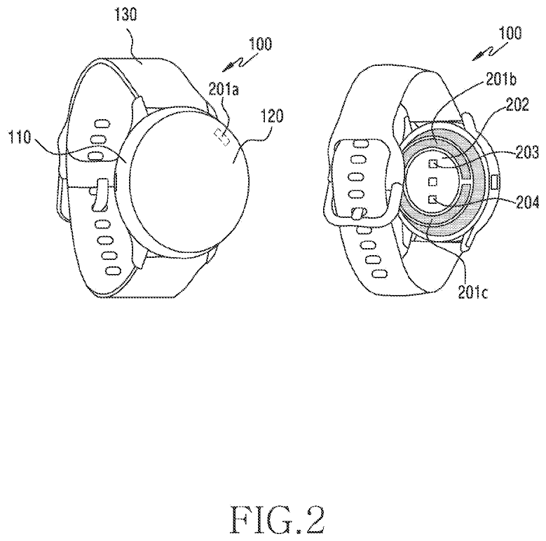

FIG. 2 is a perspective view illustrating a wearable device according to an embodiment of the disclosure.

Referring to FIG. 2, the wearable device 100 may include a housing 110, a display 120, and a strap 130. According to an embodiment, the wearable device 100 may omit at least one of the illustrated components or may additionally include other components.

According to an embodiment, the housing 110 may include a top surface, a bottom surface, and a side surface surrounding a space between the top surface and the bottom surface. According to an embodiment, the display 120 may be exposed through one area of the housing 110.

According to an embodiment, a plurality of electrodes (e.g., 201a, 201b, and 201c) may be arranged on at least a portion of the housing 110. For example, the first electrode 201a may be arranged on the top or side surface of the housing 110, and the second electrode 201b and the third electrode 201c may be arranged on the bottom surface of the housing 110. In various embodiments, the shape or size of the electrode may be configured in various ways.

According to an embodiment, a PPG sensor 202 may be exposed through the bottom surface of the housing 110. According to an embodiment, the PPG sensor 202 may include a light emitting module 203 and a light receiving module 204. According to an embodiment, the light emitting module 203 may include light emitting diodes (LEDs) having various wavelengths. For example, the light emitting module 203 may include an infrared (IR) LED, a red LED, a green LED, and/or a blue LED. According to an embodiment, the light receiving module 204 may include at least one photodiode (PD).

According to an embodiment, the display 120 may display biometric data of a user obtained through a biometric sensor. According to an embodiment, the display 120 may switch a screen output based on a user input to a portion (e.g., a bezel) of the housing 110 or an input to the display 120. For example, the display 120 may switch a watch screen to a biometric data screen (e.g., a heart rate) in response to the user input.

According to an embodiment, the strap 130 may be connected to at least a portion of the housing 110 and detachably attach the wearable device 100 to a user's body part (e.g., wrist or ankle). According to an embodiment, the user of the wearable device 100 may adjust the strap 130 to increase the degree of close contact.

The structure of the above-described wearable device 100 is in various embodiments, the wearable device 100 may be implemented differently from that of FIG. 2. The wearable device 100 may have various shapes/structures suitable for performing a method for measuring biometric data disclosed in this document.

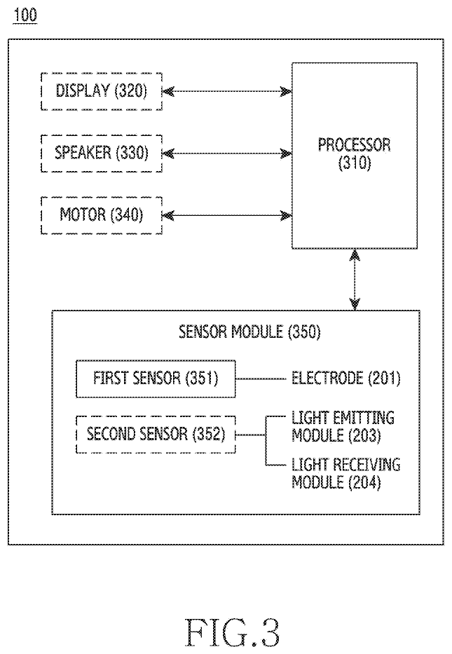

FIG. 3 is a block diagram illustrating a wearable device according to an embodiment of the disclosure.

Referring to FIG. 3, the wearable device 100 according to an embodiment may include a processor 310, a display 320, a speaker 330, a motor 340, and a sensor module 350. In various embodiments, the wearable device 100 may include additional components in addition to the components shown in FIG. 3 or may omit at least one of the components shown in FIG. 3.

According to an embodiment, the processor 310 may execute operations or data processing related to control and/or communication of at least one other component of the wearable device 100 using instructions stored in a memory (not shown). According to an embodiment, the processor 310 may include at least one of a central processing unit (CPU), a graphics processing unit (GPU), a micro controller unit (MCU), a sensor hub, a supplementary processor, a communication processor, an application processor, an application specific integrated circuit (ASIC), and a field programmable gate array (FPGA), and may have a plurality of cores.

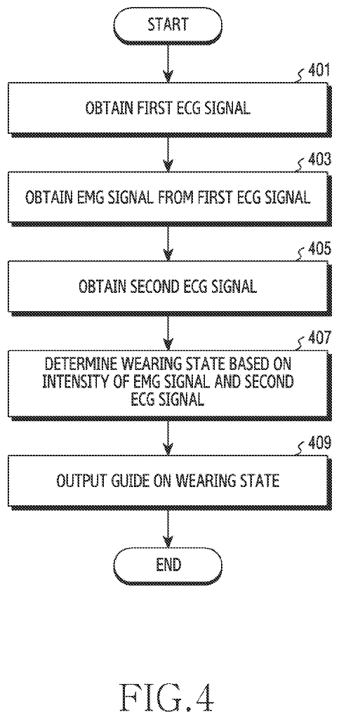

According to an embodiment, the processor 310 may obtain user's biometric information from the sensor module 350. According to an embodiment, the processor 310 may obtain a PPG signal and an ECG signal from the sensor module 350. According to an embodiment, the processor 310 may determine the wearing state of the wearable device by separating an EMG signal from the ECG signal. Details related to the operation of the processor 310 will be described later with reference to FIGS. 4 to 6.

According to an embodiment, the display 320 (e.g., the display 120 of FIG. 2) may display various contents. For example, the display 320 may display at least one of text, image, video, icon, and/or symbol. According to an embodiment, the shape of the display 320 may correspond to the shape of the housing 110. For example, the shape of the display 320 may be one of a circular shape, an elliptical shape, and a polygonal shape. However, the shape of the display 320 is not limited thereto and may have various shapes. According to an embodiment, the display 320 may be coupled to or arranged adjacent to a touch sensing circuit and/or a pressure sensor capable of measuring the intensity (pressure) of a touch. According to an embodiment, the display 320 may display biometric information of the user according to a command of the processor 310. For example, the user's biometric information may be displayed as numerical values and/or graphs. According to an embodiment, the display 320 may provide a guide on a method of measuring biometric information according to the command of the processor 310.

According to an embodiment, the speaker 330 may output various notifications to the user of the wearable device 100 as sounds (e.g., music or voice). According to an embodiment, the speaker 330 may output the user's biometric information as sound according to the command of the processor 310. For example, the user's biometric information may be output as a voice guide on numerical values and/or health state information. According to an embodiment, the speaker 330 may provide a guide on whether ECG measurement is performed and the wearing state of the wearable device by voice, according to the command of the processor 310. However, it is not limited thereto and the speaker 330 may provide various guides similar to that of the wearing state by voice.

According to an embodiment, the motor 340 may output various notifications through vibrations. For example, the motor 340 may output a notification about the start and end of ECG measurement through vibration. For example, when the user wears the wearable device 100, a guide on the start of measurement of the user's biometric information may be output through a vibration according to the command of the processor 310. In addition, the motor 340 may output a guide on the end of measurement of the user's biometric information through vibration according to the command of the processor 310. According to an embodiment, the motor 340 may output, in response to a command of the processor 310, a guide on the wearing state of the wearable device and re-measurement of biometric information through vibration.

According to an embodiment, the guide that can be output by the display 320, the speaker 330, and the motor 340 according to the command of the processor 310 may vary. The wearable device 100 may output the guides output by the display 320, the speaker 330, and the motor 340, including various other components.

According to an embodiment, the sensor module 350 may detect a user's state and provide a signal corresponding to the detected state. According to an embodiment, the sensor module 350 may include a first sensor 351 and a second sensor 352.

According to an embodiment, the first sensor 351 may include at least one of an electrocardiogram (ECG) sensor, an electrodermal activity (EDA) sensor, an electroencephalography (EEG) sensor, and a bioelectrical impedance analysis (BIA) sensor. According to an embodiment, the first sensor 351 may be electrically connected to the plurality of electrodes 201 (e.g., the first electrode 201a, the second electrode 201b, or the third electrode 201c of FIG. 2).

According to an embodiment, the plurality of electrodes 201 included in the first sensor 351 may be arranged in various positions of the wearable device 100. For example, the first electrode 201a may be arranged on the bottom surface of the housing 110 and the second electrode 201b may be arranged on the side surface or top surface of the housing 110. According to an embodiment, the first electrode 201a may be arranged on the display 320 and the second electrode 201b may be arranged on a portion of the housing 110. According to one embodiment, the first to third electrodes 201a to 201c are not limited to the described example and may be replaced with each other.

According to an embodiment, the second sensor 352 may include a PPG sensor (e.g., the PPG sensor 202 of FIG. 2). The second sensor 352 may include a light emitting module 203 and a light receiving module 204. According to an embodiment, a signal processing module (not shown) may control the light emitting module 203 and the light receiving module 204. According to an embodiment, the signal processing module may include a sensor driver controller that directly controls a sensor and an analog to digital converter (ADC). According to an embodiment, the signal processing module may further include other components not shown in FIG. 3 (e.g., an amplifier and/or a filter). According to an embodiment, the signal processing module may be implemented as a microprocessor.

According to an embodiment, the signal processing module may drive at least one LED of the light emitting module 203. According to an embodiment, the signal processing module may process (e.g., amplify and/or filter) a signal sensed by the light receiving module 204. For example, the signal processing module may convert a current signal sensed by the light receiving module 204 into a voltage signal, and convert the processed voltage signal into a digital signal.

According to an embodiment, the processor 310 may obtain PPG signal data through the second sensor 352. The processor 310 may measure a pulse wave using the PPG signal data. For example, the processor 310 may detect and measure, in an optical sensor, a change in optical characteristics, including a reflection and/or absorption/transmission ratio of a biological tissue, which appears when a corresponding volume is changed through the PPG signal data. According to an embodiment, the processor 310 may measure the pulse rate through the second sensor 352.

According to an embodiment, the sensor module 350 may include various sensors other than the first sensor 351 and the second sensor 352. For example, the sensor module 350 may include at least one of an acceleration sensor, a proximity sensor, a gyro sensor, a temperature sensor, an iris sensor, a temperature/humidity sensor, an illuminance sensor, and a time of flight (TOF) sensor. According to an embodiment, the processor 310 may use various sensors included in the sensor module 350 to determine the situation or external environment of the user.

In this document, the sensor module 350 may be referred to as at least one sensor, sensor circuitry, and/or similar expressions.

According to an embodiment, the wearable device 100 may include a memory. The memory may store various data obtained or used by at least one component (e.g., a processor) of the wearable device 100. According to an embodiment, the memory may store user's personal information such as the user's age, height, and weight.

According to an embodiment, the memory may store the user's biometric data obtained by the sensor module 350. For example, the memory may store the ECG signal data or PPG signal data obtained by the sensor module 350. In addition, the memory may store EMG signal data obtained by the processor 310 based on the ECG signal. According to an embodiment, the memory may store various pieces of information about the user's wearing state of the wearable device 100.

FIG. 4 is a flowchart illustrating determining of a wearing state of a wearable device in the wearable device according to an embodiment of the disclosure.

Referring to FIG. 4, in operation 401, the processor 310 may obtain ECG signal data through an ECG sensor (e.g., the first sensor 351 of FIG. 3). The ECG signal data may be expressed as a first ECG signal. According to an embodiment, the wearable device 100 may obtain the first ECG signal through the strap 130 while being worn on a part of the user's body (e.g., wrist).

According to an embodiment, the wearable device 100 may output a guide inducing to measure biometric information through any one of the display 320, the speaker 330, and the motor 340. According to another embodiment, the processor 310 may measure the ECG signal in a background method without providing a notification when the user measures the ECG signal.

According to an embodiment, the processor 310 may obtain an EMG signal from a first ECG signal in operation 403. For example, the processor 310 may obtain the EMG signal included in the first ECG signal. According to an embodiment, the processor 310 may compare the frequency band of the EMG signal with the frequency band of the ECG signal using an algorithm, and obtain the EMG signal. For example, the processor 310 may use a filtering technique (high-pass filter (HPF)) that separates a higher EMG signal frequency band (50 Hz to 150 Hz) than the ECG signal frequency band (e.g., 0.5 Hz to 40 Hz). According to an embodiment, the processor 310 may use various signal separation techniques (e.g., least mean square (LMS) or root mean square (RMS)) to separate components of a low frequency band (e.g., 0 Hz to 50 Hz) among components constituting the EMG signal.

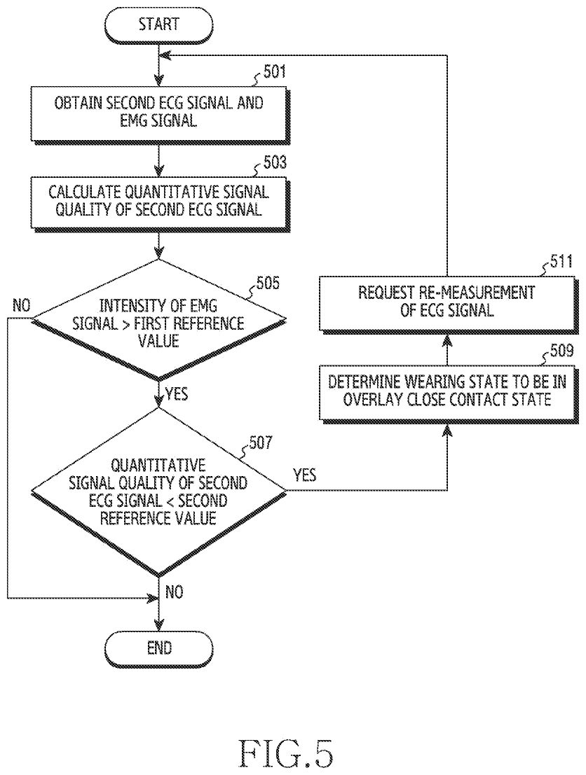

According to an embodiment, the processor 310 may obtain a second ECG signal obtained by separating the EMG signal from the first ECG signal in operation 405. For example, the processor 310 may separate the EMG signal from the first ECG signal. The separating of the EMG signal from the first ECG signal will be described with reference to FIG. 5.

According to another embodiment, in operation 405, the processor 310 may obtain a second ECG signal by separating the EMG signal from the first ECG signal and then removing noise. For example, the processor 310 may remove noise mixed with the EMG signal from the filtered EMG signal.

In operation 407, the processor 310 according to an embodiment may determine the wearing state of the wearable device 100 by the user based on the EMG signal and the second ECG signal. For example, in relation to the wearing state of the wearable device 100, the processor 310 may determine whether the wearable device 100 is in an overly close contact state in which accurate electrocardiogram measurement and photoplethysmogram measurement are impossible.

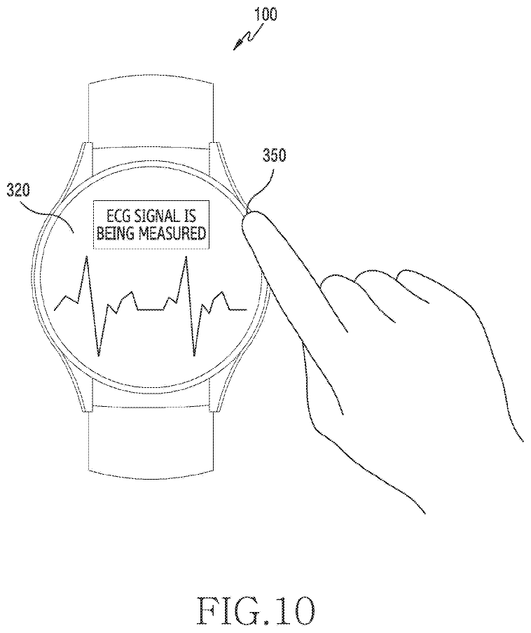

According to an embodiment, in operation 409, the wearable device 100 may output a guide on the wearing state of the wearable device 100. For example, the wearable device 100 may output various guides on whether the wearable device 100 is in the overly close contact state or whether the wearable device 100 is shaking. Details of the guide on the wearing state will be described in detail with reference to FIGS. 10 to 12.

FIG. 5 is a flowchart illustrating determining of a wearing state of a wearable device in the wearable device according to an embodiment of the disclosure.

Referring to FIG. 5, in operation 501, the processor 310 may obtain an EMG signal by separating the EMG signal from the first ECG signal described with reference to FIG. 4. According to an embodiment, the processor 310 may obtain a second ECG signal obtained by filtering the EMG signal from the first ECG signal. According to another embodiment, the processor 310 may obtain the second ECG signal obtained by filtering the EMG signal and noise from the first ECG signal.

According to an embodiment, in operation 501, the processor 310 may use a plurality of filter models having different frequency bands to obtain the second ECG signal and the EMG signal. For example, the processor 310 may obtain the EMG signal by passing the first ECG signal through a first filter. The first ECG signal that has passed through the first filter may be a signal obtained by filtering the EMG signal. According to an embodiment, the processor 310 may pass the signal having passed through the first filter, through a second filter. According to an embodiment, the signal having passed through the second filter may be a second ECG signal obtained by filtering the EMG signal and noise from the first ECG signal. According to an embodiment, the frequency band of the filter may be configured in various ways as needed. For example, the filters may be implemented in various ways.

In operation 503, the processor 310 according to an embodiment may calculate signal quality based on the second ECG signal. For example, a quantitative signal quality (signal to noise ratio (SNR)) of the second ECG signal may be calculated. According to an embodiment, the quantitative signal quality may represent whether the quality of the collected ECG is sufficiently excellent to meet the purpose of the wearable device 100.

According to an embodiment, in operation 505, the processor 310 may compare the intensity of the EMG signal with the magnitude of a first reference value. For example, the processor 310 may determine whether the intensity of the EMG signal is inadequate for determining the user's ECG signal. According to an embodiment, when the intensity of the EMG signal is greater than or equal to the first reference value, the processor 310 may determine that the wearable device 100 of the user is in an overly close contact state. For example, when the user strongly applies force to the wearable device 100 when measuring the ECG signal, the intensity of the EMG signal included in the first ECG signal through electrical stimulation may be greater than or equal to a first reference value.

According to an embodiment, when the intensity of the EMG signal is equal to or less than the first reference value, the processor 310 may determine that the wearable device 100 is not in the overly close contact state and may terminate the determination of the wearing state of the wearable device 100. According to an embodiment, the processor 310 may perform operation 507 when the intensity of the EMG signal is equal to or greater than the first reference value.

According to an embodiment, the processor 310 may compare the quality of the second ECG signal with a second reference value in operation 507. According to an embodiment, the quality of the second ECG signal may indicate quantitative signal quality, and the first reference value and the second reference value may be different from each other. According to an embodiment, in a case in which the user measures the ECG signal and/or the photoplethysmogram, when the quality of the ECG signal is sufficiently excellent even if the user is using a lot of muscles, the processor 310 may calculate a corresponding result based on the measured signal. For example, when the quality of the second ECG signal is greater than or equal to the second reference value even if the intensity of the EMG signal is greater than or equal to the first reference value, the processor 310 may determine that the quality of the signal is sufficient for calculating the result through measurement of the user's body information including the ECG signal. Accordingly, the processor 310 may terminate the determination of the wearing state of the wearable device 100 when the quality of the second ECG signal is equal to or greater than the second reference value.

According to an embodiment, when the quality of the second ECG signal is equal to or less than the second reference value, the processor 310 may determine the wearing state of the wearable device 100 to be in the overlay close contact state in operation 509. In an embodiment, the processor 310 may request re-measurement of the ECG signal in operation 511 when it is determined that the wearing state of the wearable device 100 is poor (e.g., the overlay close contact state, etc.). For example, the processor 310 may output a guide on the overlay close contact state to the user through the display 320, the speaker 330, and/or the motor 340. Without being limited thereto, the processor 310 may provide the guide on the overly close contact state using various components included in the wearable device 100. Alternatively, when there is an external device connected to the wearable device 100, the processor 310 may transmit data related to the wearing state to the external device to provide the guide on the overly close contact state using the configuration included in the external device.

According to an embodiment, the wearable device 100 may request a guide on whether to remeasure the ECG signal. The processor 310 may start re-measuring the ECG signal based on a user's response to the guide. After operation 511, the wearable device 100 may repeatedly perform operations 501 to 509.

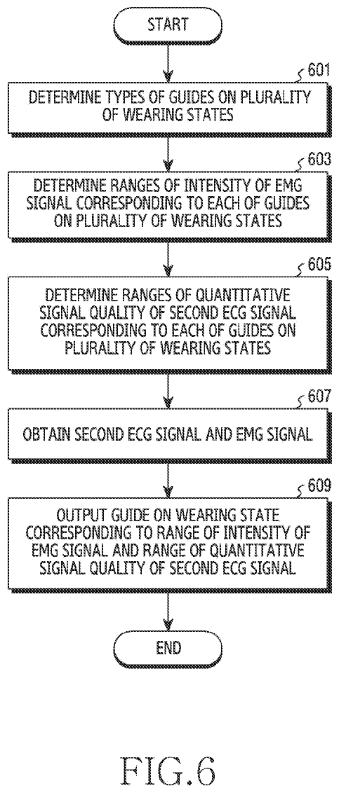

FIG. 6 is a flowchart illustrating outputting of guides on a plurality of wearing states in a wearable device according to an embodiment of the disclosure.

Referring to FIG. 6, in operation 601, the processor 310 may determine types of guides on a plurality of wearing states of the wearable device 100. For example, the type of the wearing state of the user's wearable device 100 may be determined as poor, average, good, and excellent. A poor wearing state of the wearable device 100 may indicate an inappropriate wearing state of the wearable device 100 not suitable for measuring the ECG signal and the photoplethysmogram wave. According to an embodiment, the types of the guides on the plurality of wearing states may vary.

In operation 603, the processor 310 may determine the intensity of the EMG signal corresponding to each of the guides on the plurality of wearing states. The intensity of the EMG signal may represent the intensity of the EMG signal separated from the first ECG signal described with reference to FIG. 4. The processor 310 according to an embodiment may classify the intensity of the EMG signal that can be included in the ECG signal into four ranges when there are four types of guides on the plurality of wearing states (poor, average, good, and excellent). For example, when the intensity of the EMG signal is less than or equal to a first reference value, it may correspond to excellent, when the intensity thereof exceeds the first reference value and is less than or equal to the second reference value, it may correspond to good, when the intensity thereof exceeds the second reference value and is less than or equal to a third reference value, it may correspond to average, and when the intensity thereof exceeds the third reference value, it may correspond to poor.

According to an embodiment, in operation 605, the processor 310 may determine a range of the quality of the ECG signal corresponding to each of the guides on the plurality of wearing states of the wearable device 100. According to an embodiment, the quality of the ECG signal may represent the quality of the second ECG signal described with reference to FIG. 4, and the quality of the signal may represent quantitative signal quality. For example, the second ECG signal may represent a signal obtained by filtering the EMG signal from the first ECG signal or a signal obtained by filtering the EMG signal and noise from the first ECG signal. According to an embodiment, the processor 310 may classify the range of the quality of the second ECG signal into four ranges when the guides on the plurality of wearing states is four types (poor, average, good, and excellent). For example, when a value of the quantitative signal quality of the second ECG signal is less than or equal to a fourth reference value, it may correspond to poor, when the value thereof exceeds the fourth reference value and is less than or equal to a fifth reference value, it may correspond to average, when the value thereof exceeds the fifth reference value and is less than or equal to a sixth reference value, it may correspond to good, and when the value thereof exceeds the sixth reference value, it may correspond to excellent.

According to an embodiment, the memory described with reference to FIG. 3 may store the types of the guides on the plurality of wearing states and the ranges of the intensity of the EMG signal corresponding to each of the guides on the plurality of wearing states. The memory may store the first ECG signal, the EMG signal, and the second ECG signal under the control of the processor 310.

According to an embodiment, the processor 310 may obtain the second ECG signal and the EMG signal in operation 607, similar to the operations described with reference to FIGS. 4 and 5. For example, the processor 310 may obtain ECG signal data through an ECG sensor (e.g., the first sensor 351 of FIG. 3), and perform the operation described in operation 501 of FIG. 5 to obtain the second ECG signal and the EMG signal.

In operation 609, the processor 310 according to an embodiment may determine which wearing state among the types of the guides on the plurality of wearing states corresponds to the intensity of the EMG signal and the quality of the second ECG signal. For example, when the intensity of the EMG signal is equal to or less than the first reference value and the quality of the second ECG signal is greater than the sixth reference value, the processor 310 may determine that the wearing state of the wearable device 100 is “excellent”.

According to an embodiment, in operation 609, the processor 310 may output a guide on the wearing state of the wearable device 100 based on the determination result. For example, the guide on the wearing state may be output through the display 320, the speaker 330, and/or the motor 340. According to an embodiment, the processor 310 may output the guide on the wearing state through various components of the wearable device 100.

According to an embodiment, the processor 310 may determine the wearing state by considering the intensity of the EMG signal and the quality of the second ECG signal in combination according to the performance of the wearable device 100. For example, the processor 310 may output guides on different wearing states depending on the type of the wearable device 100 even if the intensity of the EMG signal and the quality of the second ECG signal are the same.

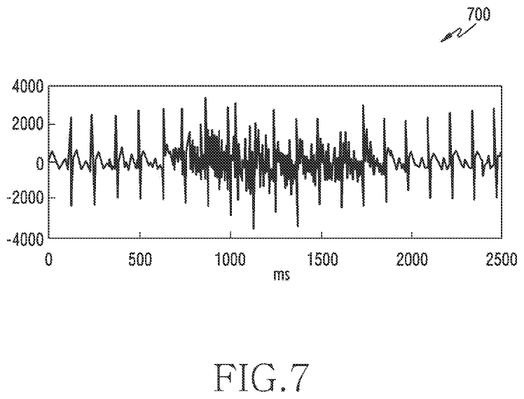

FIG. 7 is a diagram illustrating an ECG signal obtained through a wearable device according to an embodiment of the disclosure.

FIG. 7 is a graph showing an ECG signal 700 obtained by the processor 310 through an ECG sensor (e.g., the first sensor 351 of FIG. 3). The X-axis of the graph of the ECG signal 700 according to an embodiment is a time axis. In addition, the Y-axis represents the magnitude of the ECG signal over time as voltage.

According to an embodiment, the ECG signal 700 whose quality is degraded by the EMG signal may appear when the user strongly adheres the wearable device 100 the user's skin or applies strong force when measuring the ECG signal. The ECG signal 700 whose waveform is distorted by the EMG signal to degrade the signal quality may reduce the accuracy of biometric signal measurement.

The graph of the ECG signal 700 may be expressed in various ways, and is not limited to those described herein.

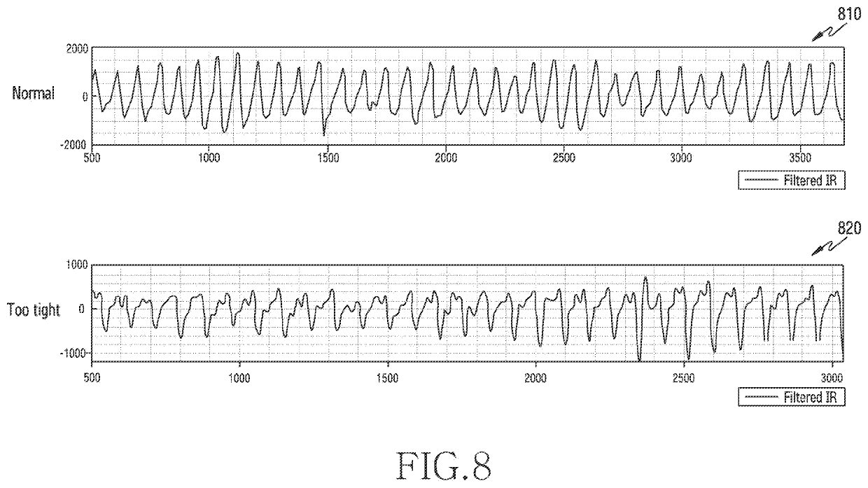

FIG. 8 is a diagram illustrating a photoplethysmography signal obtained through a wearable device according to an embodiment of the disclosure.

Referring to FIG. 8, a photoplethysmography signal (PPG signal) measured through a PPG sensor (e.g., the second sensor 352 of FIG. 3) of a wearable device according to an embodiment will be described.

The X-axis of a normal PPG signal 810 and an overly close contact PPG signal 820 according to an embodiment denotes a time axis, and the Y-axis thereof denotes the magnitude of the PPG signal as a voltage. According to an embodiment, the PPG signal measured through the PPG sensor may increase the amount of absorbed light as the amount of blood flowing through the blood vessels in the user's skin increases, and thus the intensity of the PPG signal may increase. When at least one LED of the PPG sensor emits light, some of the light may reach the user's arterial blood, venous blood, skeletal and/or skin tissue (e.g., epidermis and/or dermis). For example, some of the light reaching the arterial blood may be changed and absorbed due to a change in the arterial blood volume according to a user's pulse, and some of the light may form the PPG signal 810. The value of the PPG signal 810 may represent a difference between the user's systolic blood flow and diastolic blood flow. The PPG appears in the form of the start point of left ventricular contraction to the point of maximal contraction, the point at which contraction decreases and the expansion of the aortic wall and blood outflow decreases, and elastic waves in the peritoneum and myocardium.

The normal PPG signal 810 according to an embodiment may represent a graph in which the quality of the signal is normal when the user wears the wearable device 100 normally. The overly close contact PPG signal 820 according to an embodiment may show a graph in which the waveform of the PPG signal is distorted when the user brings the wearable device 100 into close contact or applies strong force. The PPG signal whose waveform is distorted and whose signal quality is degraded may reduce the accuracy of biometric signal measurement.

According to various embodiments, a graph of the PPG signal 810 may be generated in various ways, and is not limited to those described herein.

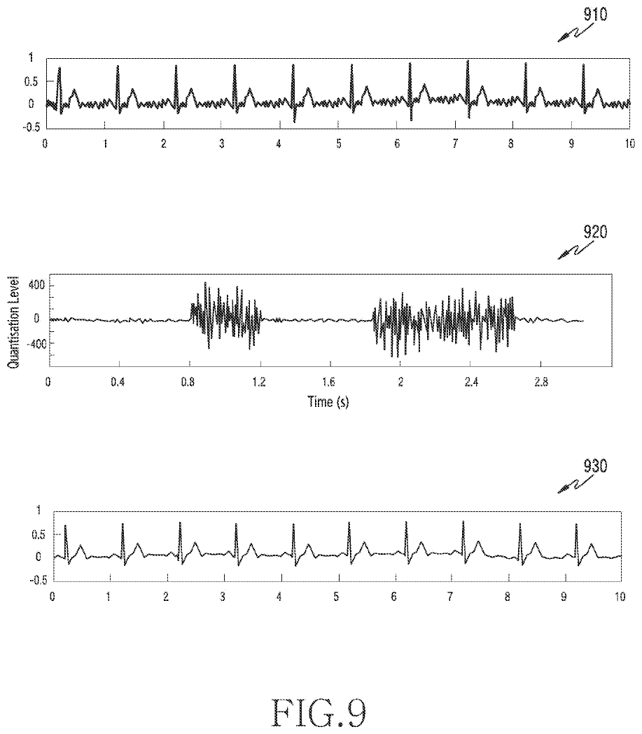

FIG. 9 is a diagram illustrating a signal obtained by separating an EMG signal from an ECG signal obtained through a wearable device according to an embodiment of the disclosure.

Referring to FIG. 9, a first ECG signal 910 is a graph representing an ECG signal obtained by the processor 310 through an ECG sensor (e.g., the first sensor 351 of FIG. 3). An EMG signal 920 according to an embodiment is a graph representing an EMG signal separated from the first ECG signal 910 by the processor 310. According to an embodiment, the second ECG signal 930 is a graph representing a signal obtained by filtering the EMG signal from the first ECG signal 910 by the processor 310.

According to an embodiment, the first ECG signal 910, the EMG signal 920, and/or the second ECG signal 930 may correspond to the first ECG signal, the EMG signal, and/or the second ECG signal described with reference to FIGS. 4 to 5, respectively.

The X-axis of the first ECG signal 910, the EMG signal 920, and the second ECG signal 930 is a time axis, and the Y-axis thereof represents the magnitude of each signal as a voltage.

According to various embodiments, the graphs for signals 910, 920, and 930 described with reference to FIG. 9 may be generated in various ways, and are not limited to those described herein.

FIG. 10 is a diagram illustrating an ECG measurement UI displayed on a display in a wearable device according to an embodiment of the disclosure.

Referring to FIG. 10, the wearable device 100 according to an embodiment may measure user's biometric information through an ECG sensor and a PPG sensor (sensor module 350). According to an embodiment, when it is determined that the user's biometric information is normally measured, the processor 310 may output a guide message through the display 320. For example, the processor 310 may output a guide message (e.g., “ECG signal is being measured”) to notify that the user's biometric information is being normally measured.

According to one embodiment, the processor 310 may simultaneously display the ECG signal and the PPG signal through the display 320. For example, the processor 310 may display the ECG signal and the PPG signal numerically and/or graphically.

According to the above-described embodiment, the wearable device 100 may support the user to intuitively recognize the measurement result by providing the user's biometric information through a UI including numerical values and/or graphs.

According to the above-described embodiment, the wearable device 100 indicates that the biometric information is normally measured through the display 320, but is not limited to the display 320. The guide may be output through various components of the wearable device 100 (e.g., the speaker 330 and the motor 340) without being limited to the display 320.

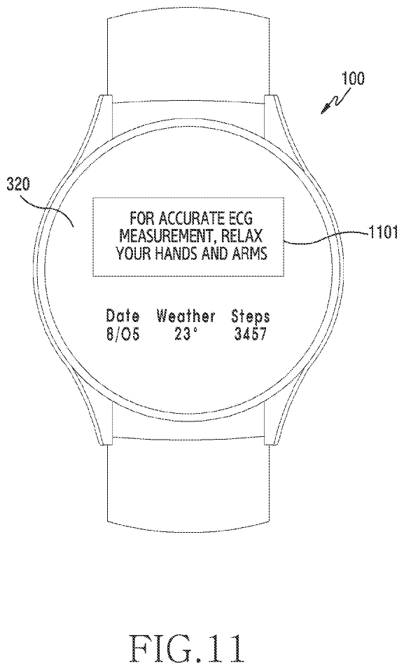

FIG. 11 is a diagram illustrating a wearing state guide UI displayed on a display in a wearable device according to an embodiment of the disclosure.

Referring to FIG. 11, the wearable device 100 according to an embodiment may output a guide on the wearing state determined based on user's biometric information (e.g., an ECG signal, etc.) obtained through the sensor module 350. For example, the processor 310 may determine the user's wearing state of the wearable device 100 based on the operations described with reference to FIGS. 4 and 5. As a result of the determination, the processor 310 may output a guide message 1101 through the display 320 when it is determined that the wearing state is inappropriate for measuring the user's body information (e.g., an ECG signal, etc.) (e.g., an overly close contact state).

According to another embodiment, the processor 310 may output a voice guide or sound through the speaker 330 to output a guide on the wearing state. For another example, the processor 310 may provide vibration through the motor 340 in order to output the guide on the wearing state. According to an embodiment, the processor 310 may output at least two of a UI, sound, or vibration.

According to an embodiment, the wearable device 100 may provide a remeasurement guide by outputting at least one of the UI, sound, and vibration. For example, when it is determined that the user's wearing state of the wearable device 100 is inappropriate (e.g., overly close contact state), the processor 310 may provide a biometric signal re-measurement guide and re-measure the user's biometric information (e.g., ECG signal).

According to an embodiment, when the sensor module 350 is not in close contact with the user's skin or when the sensor module 350 is excessively close to the user's skin to deteriorate the biosignal quality, the wearable device 100 may induce the user to change the wearing state to an appropriate close state through the guide message 1101.

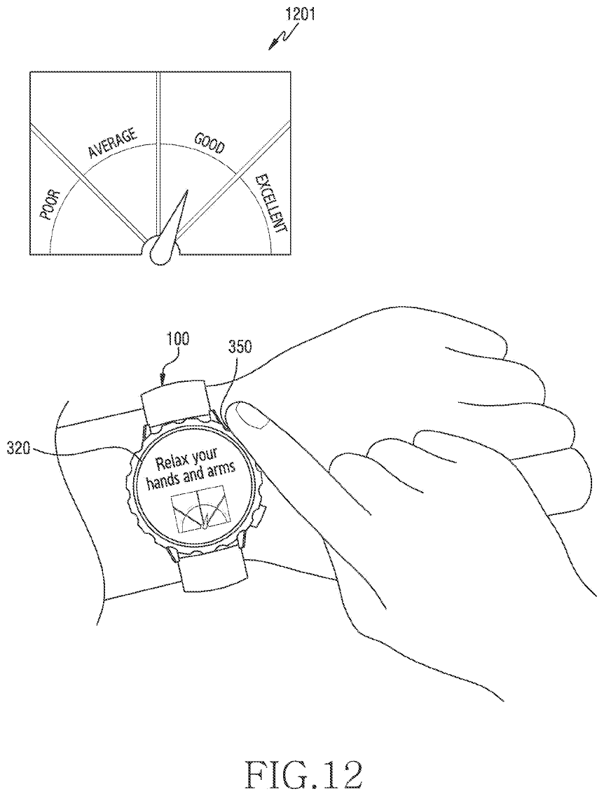

FIG. 12 is a diagram illustrating a plurality of guide UIs for a wearing state displayed on a display in a wearable device according to an embodiment of the disclosure.

Referring to FIG. 12, the wearable device 100 according to an embodiment may output a plurality of guides on the wearing state determined based on user's biometric information (e.g., an electrocardiogram signal, etc.) obtained through the sensor module 350. For example, the processor 310 may determine the user's wearing state of the wearable device 100 based on the operation described with reference to FIG. 6. According to an embodiment, the processor 310 may output guide messages corresponding to the guides on the plurality of wearing states described with reference to FIG. 6 based on the determination result. The guide message is not limited in expression and may indicate data similar to the guide on the wearing states provided in various forms. For example, the processor 310 may output, through the display 320, a picture or video as the guide on the user's wearing state of the wearable device 100. According to an embodiment, the processor 310 may determine the wearing state in response to the degree of adhesion of the sensor module 350 to the user's skin, and output a signal quality determination result based on the current wearing state. For example, the processor 310 may output the signal quality determination result using at least one visual object according to the degree of signal quality. For example, the processor 310 may output the signal quality determination result based on the current wearing state using an animation such as a traffic light or a gauge, as illustrated by element 1201.

According to an embodiment, at least one of the plurality of electrodes 201 for obtaining the user's biometric signal may be disposed on the display 320. The user may measure the biometric signal by applying force on the display 320 with the hand opposite to the hand wearing the wearable device 100. According to an embodiment, the user may naturally apply force to the display 320. Based on the force applied to the display 320, the processor 310 according to an embodiment may determine whether the display 320 is in an inappropriate wearing state for measuring biometric information due to an overlay close contact state. For example, when measuring the ECG signal, the processor 310 may determine whether the user exerts excessive force on the display 320 and the ECG signal is mixed so that precise measurement is difficult.

The wearable device 100 according to an embodiment may guide the wearing state of the wearable device 100 for measuring biometric information and guide a wearing method for accurately measuring biometric information. For example, the wearable device 100 may guide an immediate change in the wearing state using a UI, sound, and vibration. The wearable device 100 may induce the user to learn wearing know-how capable of measuring a signal with good quality by himself or herself. However, the wearable device 100 is not limited to the above examples and may guide various information similar to a wearing method for accurately measuring biometric information through various methods.

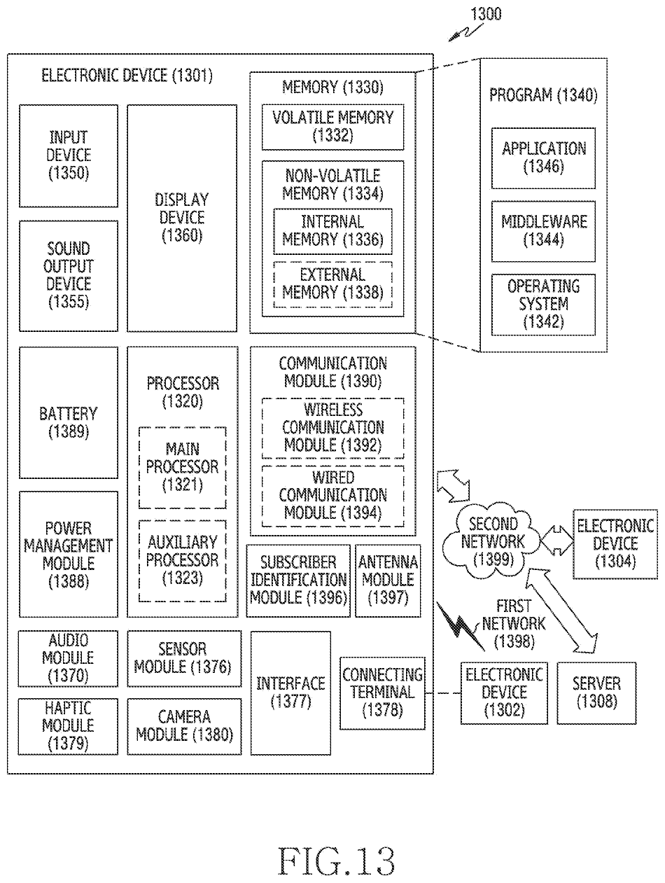

FIG. 13 is a block diagram illustrating an electronic device 1301 (e.g., the wearable device 100 of FIG. 1) in a network environment 1300 according to an embodiment of the disclosure.

Referring to FIG. 13, the electronic device 1301 in the network environment 1300 may communicate with an electronic device 1302 via a first network 1398 (e.g., a short-range wireless communication network), or at least one of an electronic device 1304 or a server 1308 via a second network 1399 (e.g., a long-range wireless communication network). According to an embodiment, the electronic device 1301 may communicate with the electronic device 1304 via the server 1308. According to an embodiment, the electronic device 1301 may include a processor 1320, memory 1330, an input module 1350, a sound output module 1355, a display module 1360, an audio module 1370, a sensor module 1376, an interface 1377, a connecting terminal 1378, a haptic module 1379, a camera module 1380, a power management module 1388, a battery 1389, a communication module 1390, a subscriber identification module (SIM) 1396, or an antenna module 1397. In some embodiments, at least one of the components (e.g., the connecting terminal 1378) may be omitted from the electronic device 1301, or one or more other components may be added in the electronic device 1301. In some embodiments, some of the components (e.g., the sensor module 1376, the camera module 1380, or the antenna module 1397) may be implemented as a single component (e.g., the display module 1360).

The processor 1320 may execute, for example, software (e.g., a program 1340) to control at least one other component (e.g., a hardware or software component) of the electronic device 1301 coupled with the processor 1320, and may perform various data processing or computation. According to one embodiment, as at least part of the data processing or computation, the processor 1320 may store a command or data received from another component (e.g., the sensor module 1376 or the communication module 1390) in volatile memory 1332, process the command or the data stored in the volatile memory 1332, and store resulting data in non-volatile memory 1334. According to an embodiment, the processor 1320 may include a main processor 1321 (e.g., a central processing unit (CPU) or an application processor (AP)), or an auxiliary processor 1323 (e.g., a graphics processing unit (GPU), a neural processing unit (NPU), an image signal processor (ISP), a sensor hub processor, or a communication processor (CP)) that is operable independently from, or in conjunction with, the main processor 1321. For example, when the electronic device 1301 includes the main processor 1321 and the auxiliary processor 1323, the auxiliary processor 1323 may be adapted to consume less power than the main processor 1321, or to be specific to a specified function. The auxiliary processor 1323 may be implemented as separate from, or as part of the main processor 1321.

The auxiliary processor 1323 may control at least some of functions or states related to at least one component (e.g., the display module 1360, the sensor module 1376, or the communication module 1390) among the components of the electronic device 1301, instead of the main processor 1321 while the main processor 1321 is in an inactive (e.g., sleep) state, or together with the main processor 1321 while the main processor 1321 is in an active state (e.g., executing an application). According to an embodiment, the auxiliary processor 1323 (e.g., an image signal processor or a communication processor) may be implemented as part of another component (e.g., the camera module 1380 or the communication module 1390) functionally related to the auxiliary processor 1323. According to an embodiment, the auxiliary processor 1323 (e.g., the neural processing unit) may include a hardware structure specified for artificial intelligence model processing. An artificial intelligence model may be generated by machine learning. Such learning may be performed, e.g., by the electronic device 1301 where the artificial intelligence is performed or via a separate server (e.g., the server 1308). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 1330 may store various data used by at least one component (e.g., the processor 1320 or the sensor module 1376) of the electronic device 1301. The various data may include, for example, software (e.g., the program 1340) and input data or output data for a command related thereto. The memory 1330 may include the volatile memory 1332 or the non-volatile memory 1334.

The program 1340 may be stored in the memory 1330 as software, and may include, for example, an operating system (OS) 1342, middleware 1344, or an application 1346.

The input module 1350 may receive a command or data to be used by another component (e.g., the processor 1320) of the electronic device 1301, from the outside (e.g., a user) of the electronic device 1301. The input module 1350 may include, for example, a microphone, a mouse, a keyboard, a key (e.g., a button), or a digital pen (e.g., a stylus pen).

The sound output module 1355 may output sound signals to the outside of the electronic device 1301. The sound output module 1355 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 1360 may visually provide information to the outside (e.g., a user) of the electronic device 1301. The display module 1360 may include, for example, a display, a hologram device, or a projector and control circuitry to control a corresponding one of the display, hologram device, and projector. According to an embodiment, the display module 1360 may include a touch sensor adapted to detect a touch, or a pressure sensor adapted to measure the intensity of force incurred by the touch.

The audio module 1370 may convert a sound into an electrical signal and vice versa. According to an embodiment, the audio module 1370 may obtain the sound via the input module 1350, or output the sound via the sound output module 1355 or a headphone of an external electronic device (e.g., an electronic device 1302) directly (e.g., wiredly) or wirelessly coupled with the electronic device 1301.

The sensor module 1376 may detect an operational state (e.g., power or temperature) of the electronic device 1301 or an environmental state (e.g., a state of a user) external to the electronic device 1301, and then generate an electrical signal or data value corresponding to the detected state. According to an embodiment, the sensor module 1376 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1377 may support one or more specified protocols to be used for the electronic device 1301 to be coupled with the external electronic device (e.g., the electronic device 1302) directly (e.g., wiredly) or wirelessly. According to an embodiment, the interface 1377 may include, for example, a high definition multimedia interface (HDMI), a universal serial bus (USB) interface, a secure digital (SD) card interface, or an audio interface.

A connecting terminal 1378 may include a connector via which the electronic device 1301 may be physically connected with the external electronic device (e.g., the electronic device 1302). According to an embodiment, the connecting terminal 1378 may include, for example, a HDMI connector, a USB connector, a SD card connector, or an audio connector (e.g., a headphone connector).

The haptic module 1379 may convert an electrical signal into a mechanical stimulus (e.g., a vibration or a movement) or electrical stimulus which may be recognized by a user via his tactile sensation or kinesthetic sensation. According to an embodiment, the haptic module 1379 may include, for example, a motor, a piezoelectric element, or an electric stimulator.

The camera module 1380 may capture a still image or moving images. According to an embodiment, the camera module 1380 may include one or more lenses, image sensors, image signal processors, or flashes.

The power management module 1388 may manage power supplied to the electronic device 1301. According to one embodiment, the power management module 1388 may be implemented as at least part of, for example, a power management integrated circuit (PMIC).

The battery 1389 may supply power to at least one component of the electronic device 1301. According to an embodiment, the battery 1389 may include, for example, a primary cell which is not rechargeable, a secondary cell which is rechargeable, or a fuel cell.

The communication module 1390 may support establishing a direct (e.g., wired) communication channel or a wireless communication channel between the electronic device 1301 and the external electronic device (e.g., the electronic device 1302, the electronic device 1304, or the server 1308) and performing communication via the established communication channel. The communication module 1390 may include one or more communication processors that are operable independently from the processor 1320 (e.g., the application processor (AP)) and supports a direct (e.g., wired) communication or a wireless communication. According to an embodiment, the communication module 1390 may include a wireless communication module 1392 (e.g., a cellular communication module, a short-range wireless communication module, or a global navigation satellite system (GNSS) communication module) or a wired communication module 1394 (e.g., a local area network (LAN) communication module or a power line communication (PLC) module). A corresponding one of these communication modules may communicate with the external electronic device via the first network 1398 (e.g., a short-range communication network, such as Bluetooth™, wireless-fidelity (Wi-Fi) direct, or infrared data association (IrDA)) or the second network 1399 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 1392 may identify and authenticate the electronic device 1301 in a communication network, such as the first network 1398 or the second network 1399, using subscriber information (e.g., international mobile subscriber identity (IMSI)) stored in the subscriber identification module 1396.

The wireless communication module 1392 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 1392 may support a high-frequency band (e.g., the mmWave band) to achieve, e.g., a high data transmission rate. The wireless communication module 1392 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 1392 may support various requirements specified in the electronic device 1301, an external electronic device (e.g., the electronic device 1304), or a network system (e.g., the second network 1399). According to an embodiment, the wireless communication module 1392 may support a peak data rate (e.g., 20 Gbps or more) for implementing eMBB, loss coverage (e.g., 1364 dB or less) for implementing mMTC, or U-plane latency (e.g., 0.5 ms or less for each of downlink (DL) and uplink (UL), or a round trip of 13 ms or less) for implementing URLLC.

The antenna module 1397 may transmit or receive a signal or power to or from the outside (e.g., the external electronic device) of the electronic device 1301. According to an embodiment, the antenna module 1397 may include an antenna including a radiating element composed of a conductive material or a conductive pattern formed in or on a substrate (e.g., a printed circuit board (PCB)). According to an embodiment, the antenna module 1397 may include a plurality of antennas (e.g., array antennas). In such a case, at least one antenna appropriate for a communication scheme used in the communication network, such as the first network 1398 or the second network 1399, may be selected, for example, by the communication module 1390 (e.g., the wireless communication module 1392) from the plurality of antennas. The signal or the power may then be transmitted or received between the communication module 1390 and the external electronic device via the selected at least one antenna. According to an embodiment, another component (e.g., a radio frequency integrated circuit (RFIC)) other than the radiating element may be additionally formed as part of the antenna module 1397.

According to various embodiments, the antenna module 1397 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 1301 and the external electronic device 1304 via the server 1308 coupled with the second network 1399. Each of the electronic devices 1302 or 1304 may be a device of a same type as, or a different type, from the electronic device 1301. According to an embodiment, all or some of operations to be executed at the electronic device 1301 may be executed at one or more of the external electronic devices 1302 or 1304, or the server 1308. For example, if the electronic device 1301 should perform a function or a service automatically, or in response to a request from a user or another device, the electronic device 1301, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 1301. The electronic device 1301 may provide the outcome, with or without further processing of the outcome, as at least part of a reply to the request. To that end, a cloud computing, distributed computing, mobile edge computing (MEC), or client-server computing technology may be used, for example. The electronic device 1301 may provide ultra low-latency services using, e.g., distributed computing or mobile edge computing. In another embodiment, the external electronic device 1304 may include an internet-of-things (IoT) device. The server 1308 may be an intelligent server using machine learning and/or a neural network. According to an embodiment, the external electronic device 1304 or the server 1308 may be included in the second network 1399. The electronic device 1301 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 1340) including one or more instructions that are stored in a storage medium (e.g., internal memory 1336 or external memory 1338) that is readable by a machine (e.g., the electronic device 1301). For example, a processor (e.g., the processor 1320) of the machine (e.g., the electronic device 1301) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a non-transitory machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

As described above, a wearable device (e.g., the electronic device 100 of FIG. 1) may include a first sensor (e.g., the first sensor 351 of FIG. 3) having a plurality of electrodes (e.g., the electrode 201 of FIG. 3), and at least one processor (e.g., the processor 310 of FIG. 3) configured to be electrically connected to the first sensor, wherein the at least one processor may be configured to obtain a first ECG signal using the first sensor while the wearable device is worn on a user's body, obtain an EMG signal from the first ECG signal, obtain a second ECG signal by filtering the EMG signal from the first ECG signal, determine a wearing state of the wearable device based on an intensity of the EMG signal and a quality of the second ECG signal, and output a guide on the wearing state based on a result of the determination.

According to an embodiment, the wearable device 100 may include a display and a housing surrounding the display, a first electrode among the plurality of electrodes may be arranged on a bottom surface of the housing, and a second electrode may be arranged on a side surface or top surface of the housing.

The first electrode of the wearable device 100 according to an embodiment may be arranged on the display, and the second electrode may be arranged on a portion of the housing surrounding the display.

According to an embodiment, when the wearable device 100 obtains the second ECG signal, the at least one processor may filter the EMG signal from the first ECG signal, and may obtain the second ECG signal by removing noise in addition to the EMG signal from the signal obtained by filtering the EMG signal.

According to an embodiment, when the intensity of the EMG signal is greater than or equal to a first reference value and the quality of the second ECG signal is less than or equal to a second reference value, the at least one processor of the wearable device 100 may output a message indicating that the wearing state is an overly close contact state.

The at least one processor of the wearable device 100 according to an embodiment may output a message indicating that the wearing state is the overly close contact state and a re-measurement request message, obtain a third ECG signal using the first sensor while the wearable device is worn on the user's body based on a response to the re-measurement request message, obtain a second EMG signal from the third ECG signal, obtain a fourth ECG signal obtained by filtering the second EMG signal from the third ECG signal, determine a re-wearing state of the wearable device based on the intensity of the second EMG signal and the quality of the fourth ECG signal, and output a guide on the re-wearing state based on the determination result.

The at least one processor of the wearable device 100 according to an embodiment may determine a range of an intensity of the different EMG signals corresponding to each of guides on a plurality of wearing states and a range of the quality of the second ECG signal, and output one of the guides on the plurality of wearing states corresponding to the range of the intensity of the different ECG signals and the range of the quality of the second ECG signal.

The at least one processor of the wearable device 100 according to an embodiment may include a memory configured to store at least one of the first ECG signal, the EMG signal, the second ECG signal, the range of the intensity of the different EMG signals corresponding to each of the guides on the plurality of wearing states, and the range of the quality of the second ECG signal.

According to an embodiment, the wearable device 100 may further include a second sensor having a light emitting module and a light receiving module, and the at least one processor may obtain a PPG signal using the second sensor while the wearable device is worn on the user's body.

According to an embodiment, the wearable device 100 may further include a display, and the at least one processor may provide a notification of the wearing state through the display.

The wearable device 100 according to an embodiment may further include a motor, and the at least one processor may output vibration through the motor to provide a notification of the wearing state.

In an embodiment, the wearable device 100 may further include a speaker, and the at least one processor may output a voice guide or sound through the speaker to provide a notification of the wearing state.

As described above, a method of operating the wearable device 100 according to an embodiment may include obtaining a first ECG signal using a first sensor while the wearable device is worn on a user's body, obtaining an EMG signal from the first ECG signal, obtaining a second ECG signal by filtering the EMG signal from the first ECG signal, and determining a wearing state of the wearable device based on an intensity of the EMG signal and a quality of the second ECG signal.

The wearable device 100 according to an embodiment may include a display and a housing surrounding the display, the first sensor may include a plurality of electrodes, a first electrode among the plurality of electrodes may be arranged on a bottom surface of the housing, and a second electrode may be arranged on a side surface or top surface of the housing.

The first electrode of the wearable device 100 according to an embodiment may be arranged on the display, and the second electrode may be arranged on a portion of the housing.

In the method of operating the wearable device 100, the obtaining of the second ECG signal may further include passing the first ECG signal through a first filter, and passing the signal having passed through the first filter, through a second filter, and the first filter and the second filter may have different frequency bands.

In the method of operating the wearable device 100, the determining of the wearing state may further include determining whether the intensity of the EMG signal is greater than or equal to a first reference value, and determining whether the quality of the second ECG signal is less than or equal to a second reference value.

The method of operating the wearable device 100 according to an embodiment may further include outputting a guide on the wearing state based on a result of the determining of the wearing state.

According to an embodiment, in the method of operating the wearable device 100, the determining of the wearing state may further include determining a range of the intensity of the different EMG signals corresponding to each of the guides on the plurality of wearing states and a range of the quality of the second ECG signal, and determining one of the plurality of wearing states corresponding to the range of the intensity of the EMG signal and the range of the quality of the second ECG signal.

The outputting of the guide on the wearing state of the wearable device 100 may include outputting a message indicating that the wearing state is an overly close contact state as at least one of sound, vibration, and a screen.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable device comprising:
a first sensor having a plurality of electrodes; and
at least one processor configured to be electrically connected to the first sensor,
wherein the at least one processor is configured to:
obtain a first electrocardiogram (ECG) signal using the first sensor while the wearable device is worn on a user's body,
obtain an electromyograph (EMG) signal from the first ECG signal,
obtain a second ECG signal by filtering the EMG signal from the first ECG signal,
determine a wearing state of the wearable device based on an intensity of the EMG signal and a quality of the second ECG signal, and
output a guide on the wearing state based on a result of the determination.

2. The wearable device of claim 1, further comprising:
a display; and
a housing configured to surround the display,
wherein a first electrode among the plurality of electrodes is arranged on a bottom surface of the housing, and
wherein a second electrode is arranged on a side surface or top surface of the housing.

3. The wearable device of claim 1, wherein the at least one processor is further configured to, in case that the wearable device obtains the second ECG signal:
filter the EMG signal from the first ECG signal, and
remove noise in addition to the EMG signal from the signal obtained by filtering the EMG signal, so as to obtain the second ECG signal.

4. The wearable device of claim 1, wherein the at least one processor of the wearable device is further configured to, in case that the intensity of the EMG signal is greater than or equal to a first reference value and the quality of the second ECG signal is less than or equal to a second reference value, output a message indicating that the wearing state is an overly close contact state.

5. The wearable device of claim 4, wherein the at least one processor is further configured to:
output a message indicating that the wearing state is the overly close contact state and a re-measurement request message,
obtain a third ECG signal using the first sensor while the wearable device is worn on the user's body based on a response to the re-measurement request message,
obtain a second EMG signal from the third ECG signal,
obtain a fourth ECG signal by filtering the second EMG signal from the third ECG signal,
determine a re-wearing state of the wearable device based on an intensity of the second EMG signal and a quality of the fourth ECG signal, and
output a guide on the re-wearing state based on the determination result.

6. The wearable device of claim 5, wherein the at least one processor is further configured to:
determine a range of an intensity of different EMG signals corresponding to each of guides on a plurality of wearing states and a range of the quality of the second ECG signal, and
output one of the guides on the plurality of wearing states corresponding to the range of the intensity of different ECG signals and the range of the quality of the second ECG signal.

7. The wearable device of claim 6, wherein the at least one processor further comprises a memory configured to store at least one of the first ECG signal, the EMG signal, the second ECG signal, the range of the intensity of the different EMG signals corresponding to each of the guides on the plurality of wearing states, and the range of the quality of the second ECG signal.

8. The wearable device of claim 1, further comprising:
a second sensor having a light emitting module and a light receiving module,
wherein the at least one processor is further configured to obtain a photoplethysmography (PPG) signal using the second sensor while the wearable device is worn on the user's body.

9. The wearable device of claim 4, further comprising:
a display, wherein the at least one processor is further configured to provide a notification of the wearing state through the display.

10. A method of operating a wearable device, the method comprising:
obtaining a first ECG signal using a first sensor while the wearable device is worn on a user's body;
obtaining an EMG signal from the first ECG signal;
obtaining a second ECG signal by filtering the EMG signal from the first ECG signal; and
determining a wearing state of the wearable device based on an intensity of the EMG signal and a quality of the second ECG signal.

11. The method of claim 10,
wherein the wearable device comprises a display and a housing surrounding the display,
wherein the first sensor comprises a plurality of electrodes,
wherein a first electrode among the plurality of electrodes is arranged on a bottom surface of the housing, and
wherein a second electrode is arranged on a side surface or top surface of the housing.

12. The method of claim 11, wherein the first electrode is arranged on the display, and the second electrode is arranged on a portion of the housing.

13. The method of claim 10,
wherein the obtaining of the second ECG signal comprises:
passing the first ECG signal through a first filter; and
passing the signal having passed through the first filter, through a second filter, and
wherein the first filter and the second filter have different frequency bands.

14. The method of claim 10, wherein the determining of the wearing state comprises:
determining whether the intensity of the EMG signal is greater than or equal to a first reference value; and
determining whether the quality of the second ECG signal is less than or equal to a second reference value.

15. The method of claim 10, further comprising:
outputting a guide on the wearing state based on a result of the determining of the wearing state.
